(19) United States
(12) Patent Application Publication
(10) Pub. No.: US 2008/0294229 A1
(43) Pub. Date: Nov. 27, 2008

(54) Helical Electrodes for Intramyocardial Pacing and Sensing

(76) Inventors: Paul A. Friedman, Rochester, MN (US); Charles J. Bruce, Rochester, MN (US); Samuel J. Asirvatham, Rochester, MN (US)

Correspondence Address: FISH & RICHARDSON P.C., PO BOX 1022, MINNEAPOLIS, MN, 55440-1022, US

(21) Appl. No.: 12/131,756

(22) Filed: Jun. 2, 2008

(30) Foreign Application Priority Data
Oct. 17, 2006 (US) PCT/US2006/040613

(51) Int. Cl. A61N 1/05

(52) U.S. Cl. 607/127

(57) ABSTRACT

The invention provides for an electrical lead, a steerable sheath and steerable catheter, a sheath and catheter that attach to cardiac tissue via an anchor screw, and a method of pacing, particularly via the atrioventricular septum.

### CROSS REFERENCE TO RELATED APPLICATIONS

This application claims priority to PCT/US2006/040613 filed on Oct. 17, 2006, which claims priority to U.S. Application No. 60/753,098 filed on Dec. 22, 2005.

### TECHNICAL FIELD

This invention relates to cardiology, and more particularly to cardiac electrodes.

### BACKGROUND

Various leads have been developed for pacing the different chambers of the heart. Leads are electrical conductors, and often are coated with an outer polymeric covering. The electrical conductors in a pacing lead can be arranged linearly or co-axially.

### SUMMARY

The invention provides for 1) a bipolar helical pacing lead; 2) a steerable sheath or catheter that can be attached to cardiac tissue via an anchor screw, which can be used to maintain the placement of the sheath or catheter, respectively, at a specific intracardiac location; and 3) a method of pacing the right and left ventricles from the atrial-ventricular septum.

This invention overcomes limitations with current leads and lead delivery systems. These limitation include the inability to precisely steer, navigate, and actively fix a lead to a desired specific anatomic location; the propensity of current leads to reject far-field electrical signals (e.g., from cardiac structures adjacent to the structure in which the lead is positioned); and the inability of current leads to selectively capture intramyocardial tissue, preventing undesirable stimulation of surrounding tissue.

The capability of the present invention to overcome these limitations permits a novel form of pacing (biventricular pacing via the right atrium alone), and enhances many current types of pacing and sensing, particularly those associated with implantable defibrillators. Additionally, the capability to target and fix a therapeutic delivery tool at a precise location within the tissue has many applications outside of cardiovascular medicine.

In one aspect, the invention provides an electrical lead. Such an electrical lead generally includes a lead body and at least two electrodes. Typically, the lead body has a proximal end and a distal end, and has at least two conductors. Usually, the two electrodes are disposed at the distal end of the lead body, and have a proximal end and a distal end. Generally, the proximal ends of the electrodes are electrically connected to the distal ends of the conductors. Typically, the electrodes comprise an inner electrode and an outer electrode, wherein the outer electrode is helical shaped and has an inside-the-helix surface and an outside-the-helix surface.

In certain embodiments, the inner electrode is linear and can be partially coated with a non-conductive material. In other embodiments, the inner electrode is helical shaped and has an inside-the-helix surface and an outside-the-helix surface. In some embodiments, the inside-the-helix surface of the outer electrode is coated with a non-conductive material, or the outside-the-helix surface of the inner electrode is coated with a non-conductive material. In other embodiments, the outside-the-helix surface of the outer electrode is coated with a non-conductive material, or the inside-the-helix surface of the inner electrode is coated with a non-conductive material. In still other embodiments, the inner electrode and the outer electrode are co-axial at the proximal ends. The overall length of the electrical lead can be, without limitation, about 3 mm to about 7 mm. In different embodiments, the conductors are co-axial with one another, or are parallel to one another. In yet other embodiments, the inner electrode and the outer electrode are bipolar.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is an image of one embodiment of a bipolar pacing lead.

FIG. 2 is an image of another embodiment of a bipolar pacing lead.

FIG. 3 shows additional embodiments of a bipolar pacing lead.

FIG. 4 is an image of an embodiment of a bipolar lead.

FIG. 5 is an image showing an embodiment of a bipolar lead.

FIG. 6 is an image showing an embodiment of a bipolar lead.

FIG. 7 is an image showing an embodiment of a biopolar lead.

FIGS. 8A and 8B are images showing two embodiments of a conductive suture.

FIG. 9 is a schematic showing conventional systems for sensing myocardial infarction (FIG. 8A) and a novel approach using a bipolar lead as described herein (FIG. 8B).

FIG. 10 is a schematic showing different lead placements positions.

FIG. 11A is a side view of one embodiment of a steerable sheath. FIG. 11B is a cross-sectional view of the steerable sheath shown in FIG. 11A (at the dotted line).

Like reference symbols in the various drawings indicate like elements.

### DETAILED DESCRIPTION

The ability to pace the left and/or right ventricle from the right atrium would provide a number of benefits. There would be no need to cross the tri-cuspid valve with a pacing lead, preventing the problem of valvular regurgitation or damage, and permitting use in the setting of a mechanical valve. Additionally, left ventricular pacing has been shown effective in the treatment of heart failure, but deploying a lead for left ventricular pacing is technically challenging in that lead placement requires entering the coronary sinus and its tributaries or, for epicardial placement, surgical thoracotomy. Thus, the ability to pace the left ventricle in particular via a lead positioned in the right atrium would be of tremendous clinical benefit. The anatomical area best suited for this approach is the atrioventricular septum.

The atrioventricular septum is a small area where the septum of the right atrium is contiguous with the left ventricular septum. It is a unique region of the heart in which there is juxtaposition of left ventricular, right ventricular, and right atrial myocytes. By positioning a lead in this area, the lead can not only sense activity across both ventricles and the right atrium, but also permit capture of both ventricles and, if positioned properly, the right atrium. In order to accomplish this, a tool for delivery of a pacing lead to a precise location is required, and a lead capable of intramyocardial stimulation and sensing is necessary. Currently, pacing and defibrillation leads are directed to desired locations with the use of stylets that can be custom curved at the time of surgery or over guidewires when leads are placed in the coronary venous system. Active steering to permit precise intra-chamber positioning has, heretoforth, not been feasible. This disclosure describes both a tool for precise delivery of therapy (including placement of an electrical lead) and a novel pacing lead suitable for use intramyocardially.

### Electrical Lead

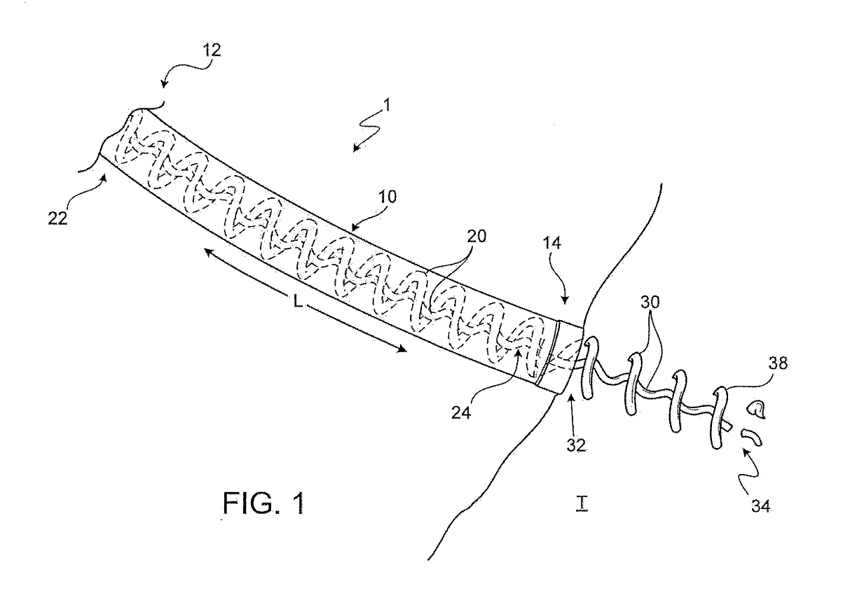

The term lead or electrical lead as used herein refers to an indewelling electrode for electrical stimulation of tissue. Electrical stimulation can be for, without limitation, pacing, defibrillation, cardiac contraction modulation, sub-threshhold stimulation, or any other type of electrical therapy. With reference to FIG. 1, an electrical lead 1 generally includes a lead body 10 having a proximal 12 and a distal 14 end. The lead body 10 typically has a lumen (l), through which at least two conductors 20 run longitudinally L. The distal end 14 of the lead body 10 has at least two electrodes 30, each having a proximal 32 and a distal 34 end. The proximal end 32 of each electrode 30 is electrically connected to the distal end 24 of a corresponding conductor 20. In some embodiments, an electrode 30 may be the exposed distal end 24 of the conductor 20.

With reference to FIGS. 1, 2 and 3, the electrical lead 1 described herein has at least an inner electrode 36 and an outer electrode 38. In some embodiments, the inner and outer electrodes can be coaxial at their proximal ends. Either or both the inner electrode 36 or the outer electrode 38 can have a helical shape. The helical shape of an electrode 30 results in the electrode having a surface that is substantially on the inside of the helix and a surface that is substantially on the outside of the helix.

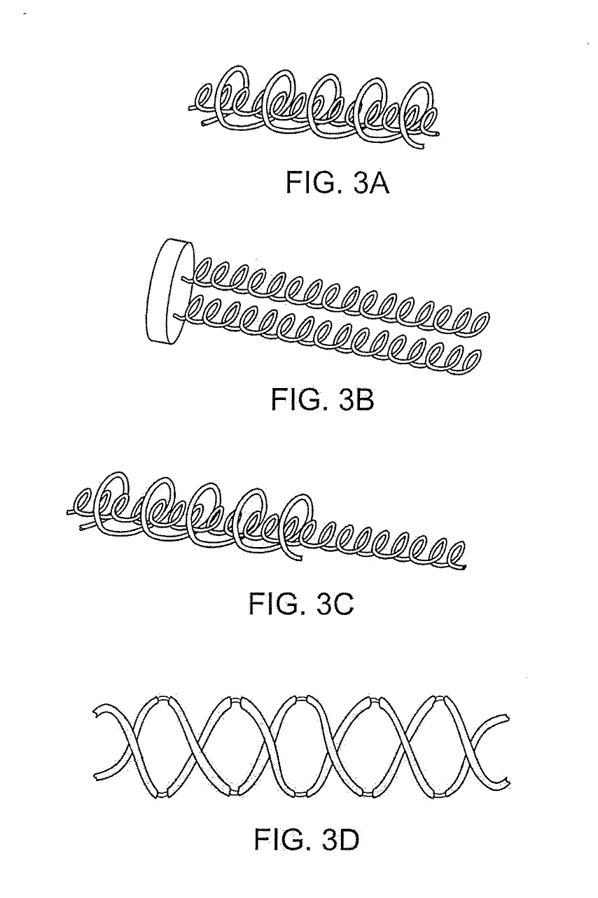

In the embodiment shown in FIG. 1, both the inner electrode 36 and the outer electrode 38 have a helical shape (i.e., a helix-within-a-helix configuration). As shown in FIG. 1, the helices of the inner electrode 36 and the outer electrode 38 need not have the same helical pitch (see, also, FIG. 3A). Although the helices in FIG. 1 are both shown as turning clockwise, either or both of the helices can be configured to turn counterclockwise. Therefore, a helix-within-a-helix configuration results in each electrode having an inside-the-helix surface and an outside-the-helix surface.

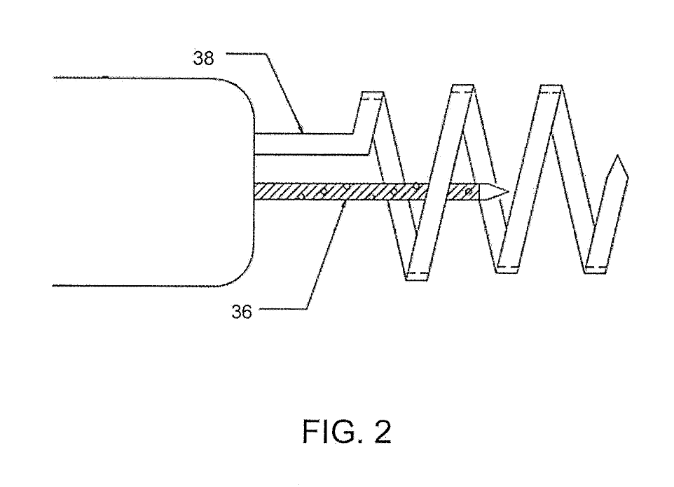

FIG. 2 shows another embodiment of an electrical lead 1 having an inner electrode 36 and an outer electrode 38. In FIG. 2, the outer electrode 38 has a helical shape while the inner electrode 36 is linear. In the embodiment shown in FIG. 2, the inner linear electrode 36 is shorter than the outer helical electrode 38. In certain applications, however, it may be desirable to have the inner linear electrode extend beyond the length of the outer helical electrode. In some embodiments, the inner linear electrode 36 can be retractable and/or deployable.

In addition to a helix-within-a-helix configuration and a linear-within-a-helix configuration, an electrical lead as described herein can have other configurations. See, for example, FIGS. 3-7 for additional embodiments of an electrical lead. For example, FIGS. 3A and 3C show electrical leads having a helix-within-a-helix configuration, in which the two helices have different pitches. In addition, an electrical lead can have two helices that have substantially the same pitch and therefore, are essentially adjacent to each other (FIG. 3B). FIG. 3D shows an embodiment similar to that shown in FIG. 1, however, in the embodiment shown in FIG. 3D, both the inner electrode and the other electrode can be entirely insulated, with the insulation removed at positions along the electrodes where the inner helix and the other helix are farthest apart. FIG. 3B shows an embodiment of a bipolar pacing lead in which both leads are helically shaped but are separated (i.e., do not have a helix-within-a-helix configuration).

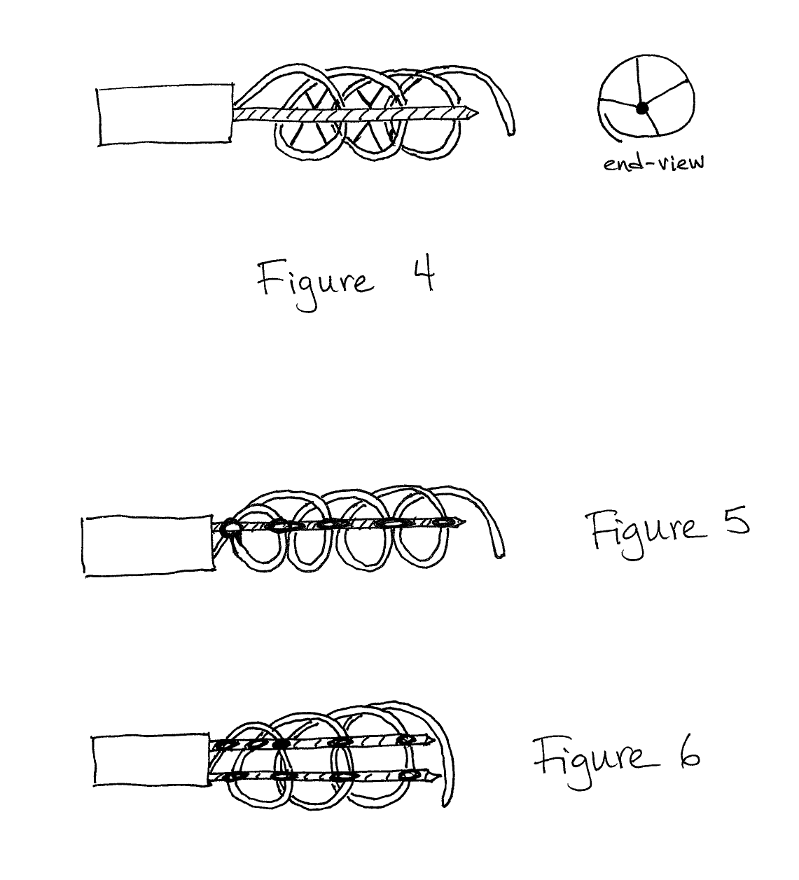

FIG. 4 demonstrates an alternative design for a linear-within-a-helix configuration. FIG. 4 shows that stabilization (or stand-off) struts can be included to prevent the inner electrode and the outer electrode from physically contacting one another. The struts can be insulated (i.e., non-conductive) and can be arranged in any pattern that accomplishes the purpose of prohibiting or reducing contact between the inner and outer electrodes. In one embodiment, the struts can be deployed after insertion of the bipolar lead to prevent coring of the cardiac tissue. The inner electrode can have an internal luman containing struts therein during deployment into the myocardium. After the inner electrode has been placed in the myocardium, the struts can be forced out of the lumen through ports/openings on the inner electrode to make contact with the outer helical electrode and provide a stand-off.

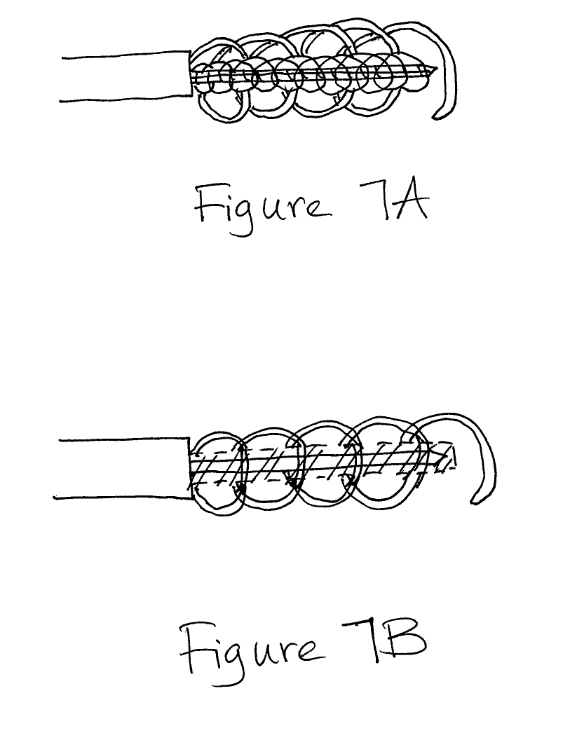

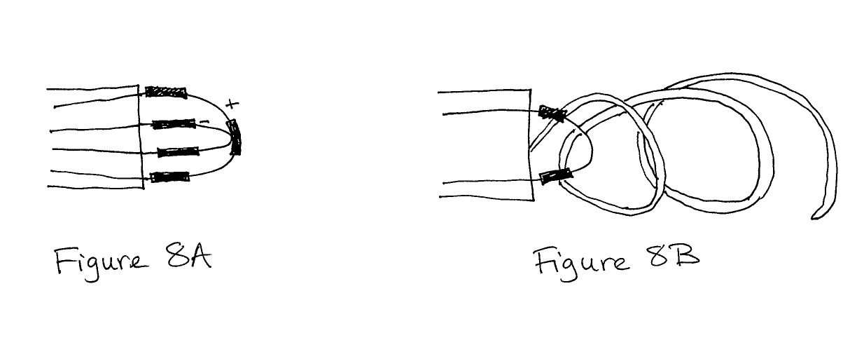

FIG. 5 shows an embodiment in which the inner electrode is positioned off-center relative to the helical outer electrode. The embodiment shown in FIG. 5 also has optional insulated rings positioned on the helical outer electrode. As discussed herein, such insulated rings can prevent the inner and outer electrodes from making contact, and such insulated rings also can be used to guide the inner electrode in an embodiment in which the inner electrode is deployable and retractable. FIG. 6 shows an embodiment that is similar to the embodiment shown in FIG. 5 but the bipolar lead shown in FIG. 6 has two inner electrodes that, in FIG. 6, are both linear. Each of the inner electrodes shown in FIG. 6 can be stabilized by insulating rings, and either or both of the inner electrodes can be deployable/retractable.

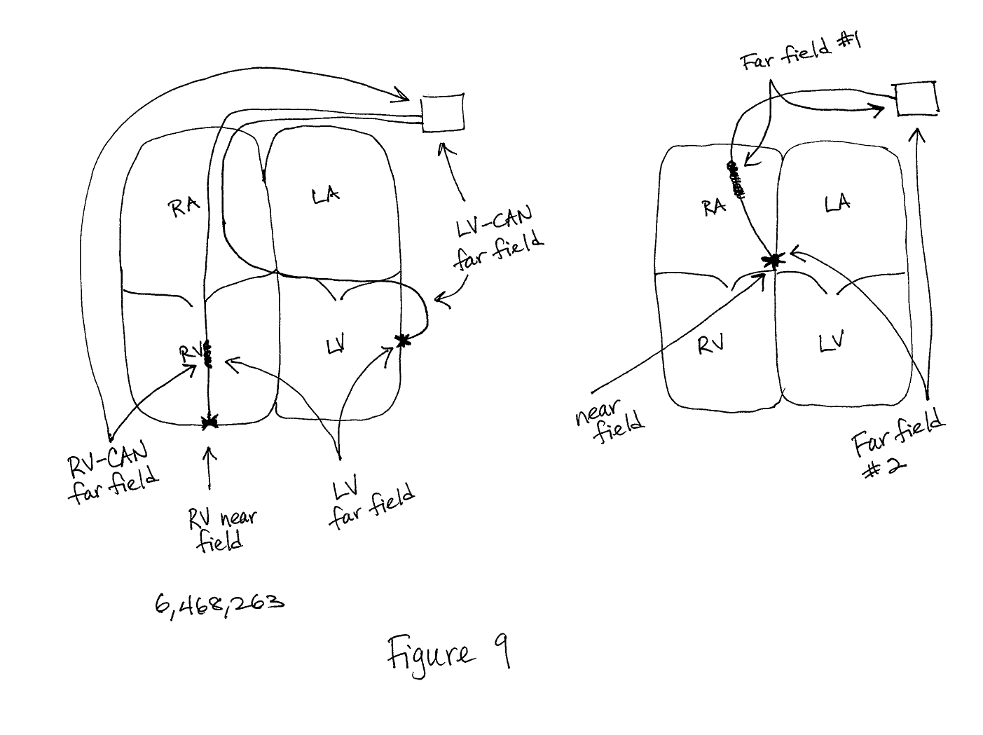

FIG. 7A shows an embodiment in which there is an inner and an outer helical electrode and a linear electrode that is within both helical electrodes. The linear electrode can be configured to prevent the inner helical electrode from making contact with the outer helical electrode (i.e., to prevent a short circuit between two electrodes). The linear electrode can be made of any non-conductive material including plastic. The embodiment shown in FIG. 7B is similar to the embodiment shown in FIG. 7A, however, the embodiment shown in FIG. 7B uses a cage-like structure to prevent the inner linear electrode from contacting the outer electrode. As indicated herein, the cage-like structure can be made from any non-conductive material such as plastic.

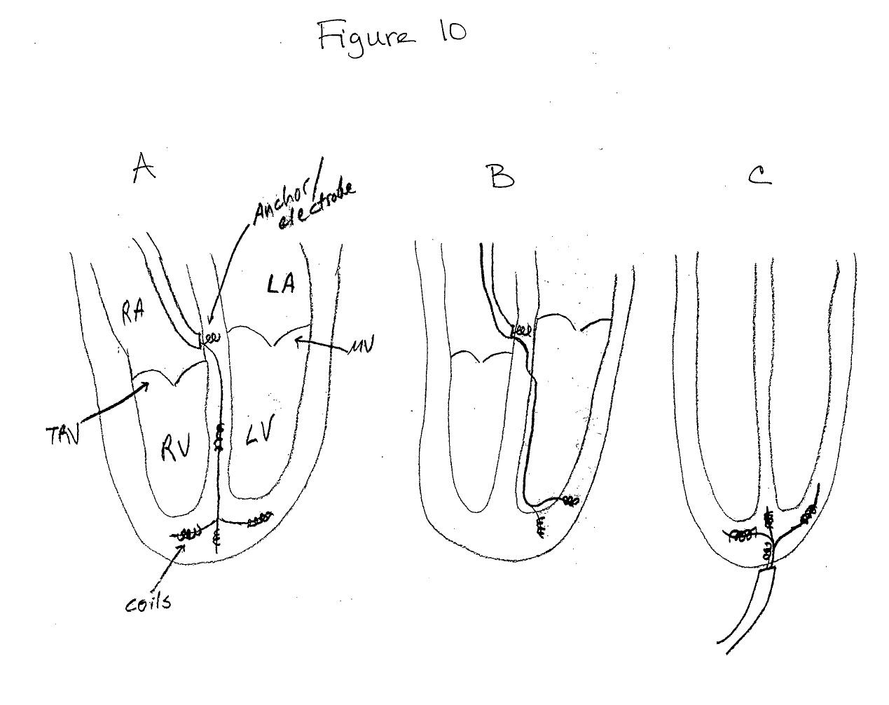

FIG. 8 shows a unique “suture lead”. As used herein, a “suture lead” is a conducting wire or thread or other like material that can be placed within the cardiac tissue and act as an electrode. Such a suture lead can be insulated at various portions along its length, which is shown in FIG. 8A, while FIG. 8B demonstrates the combination of a suture lead and a helical electrode. In this embodiment, a suture lead can be positioned inside of a helical outer electrode or a suture lead can be positioned outside of a helical inner electrode. It would be understood that one or more suture leads can be placed virtually anywhere within the cardiac tissue.

Any portion of either electrode can be insulated (e.g., by coating with a non-conductive material) to control (e.g., focus) the direction of dispersement of electromagnetic force. For example, the inside surface or the outside surface of either electrode can be insulated. In addition, either or both electrodes can be insulated in a pattern that prevents contact but allows current flow. One example is shown in FIG. 2, in which a ‘swiss-cheese’ pattern of insulation is shown, and another example is shown in FIG. 7, in which a ‘cage-like’ pattern of non-conductive material is shown. Alternatively, the openings or holes may be elongated so as to create a multi-slit or multi-slot pattern of insulation. One or more electrodes that are configured to optimize the direction of dispersement of electromagnetic force allows for a greater chance at capturing the heart.

Each electrode itself can be unipolar or bipolar, or the pair of electrodes (i.e., the electrical lead) can be unipolar or bipolar. For example, in a bipolar embodiment, the inner electrode can be the cathode and the other electrode can be the anode, or vice versa. The electrodes also can have different lengths with, for example, the inner electrode having a longer length than the outer electrode (see, for example, FIG. 3C). In one embodiment, for example, the inner electrode can be helical and have an insulator at its midpoint, while the outer electrode reaches only to the midpoint of the inner electrode. In this embodiment, the energy would flow from helix to helix where the two electrodes overlap and from the tip of the inner electrode back to the outer electrode where they don't overlap.

It is known in the art that fibrosis often develops at the site of an electrode and that long-term pacing and sensing is adversely affected by the fibrosis. This has been overcome with standard leads by using, for example, steroid-eluting tips or collars to mitigate the inflammatory response. The same or similar technology can be utilized with the leads described herein. In cases in which the insulating material coating or deposited on an electrode contains openings or cavities, steroid-containing pellets or the like can be deposited within such openings or cavities and, therefore, applied intramyocardially.

Intramyocardial pacing permits very localized cardiac excitation without far-field capture. The co-axial and helical shaped features of the electrodes described herein allow for intramyocardial pacing in thin tissue. For example, two electrodes positioned co-axially obviates the need for longer electrodes that are required by sequential electrodes. In addition, an electrode having a helical shape has a shorter effective length than its actual linear length. Therefore, a helical shaped electrode can be used to intra-myocardially stimulate thinner regions of the myocardium such as the atria or the septum.

Previously described bipolar and/or helical pacing leads would be too long and would likely perforate thinner tissues such as the septum. By way of example, a normal septum can be from about 8 mm to about 15 mm in thickness, while a diseased septum may be 6 mm to 8 mm thick. It is one object of the helical electrical lead described herein to provide a significant amount of effective surface area while having a small overall length (e.g., 3 mm, 4 mm, 5 mm, 6 mm, or 7 mm). It is noted, however, that a helical electrical lead as described herein also can be configured to have a larger overall length (e.g., 10 mm, 15 mm, 20 mm, 25 mm, 30 mm) to, for example, excite ventricular myocardium from the atrioventricular septum.

Intramyocardial pacing at the intraventricular septum can excite left-ventricular myocardium, which would permit left ventricular pacing with the lead positioned in the right atrium. Without being bound by any particular mechanism, pacing of the intraventricular septum at low output may allow for more rapid conduction to the remainder of the left ventricle (owing to the fibro-orientation at this pacing site) while pacing at a higher output may directly capture the penetrating His bundle. Either or both of these mechanisms may achieve a relatively rapid conduction to the left ventricle and, since the left ventricular myocardium is stimulated, may allow for right-sided cardiac resynchronization.

Another potential benefit of intramyocardial septal pacing is that ventricular pacing performed via the atrioventricular route avoids crossing the tricuspid valve. The presence of a lead across the tricuspid valve has been a factor associated with clinically significant tricuspid regurgitation and avoiding valve insult appears to be desirable. In addition, pacing that requires the use of a high output (e.g., cardiac contractility modulation) must capture left ventricular myocardium without phrenic nerve stimulation, which is possible using the leads described herein.

The use of small electrodes has been desirable since stimulation requires less energy, thereby saving battery life. Size reduction of electrodes, however, has been limited by diminution of the sensed electrogram as electrode size is reduced. It was determined herein that using electrodes as described herein overcomes this limitation, since acceptable electrogram amplitude and pacing thresholds were both present. The absence of stimulation of adjacent tissues with these leads might increase placement options.

It would be understood by those of skill in the art that although the electrical lead described herein is extremely well-suited for septal pacing, its use is not limited to the septum and it can be used anywhere in the heart or other tissue where electrical stimulation is required. For example, an electrical lead as described herein can be pushed through the right atrial appendage into the right ventricle, and the helical electrode can be screwed into the right ventricle for right ventricle pacing. For this application, the lead body would remain in the right atrium, but the helical electrode would be screwed through the right atrial appendage and entirely into the ventricular muscle tissue.

In one embodiment, an atrial electrode can be placed 3 to 6 cm proximal of an helical lead. After positioning the helical electrode at the tip of the lead in the septum, the body of the lead can be placed such that the atrial electrode rests firmly against the lateral wall of the right atrium just anterior to the crista terminales. Using an atrial electrode that is proximal to the helical electrode and that rests against the atrium wall can allow sensing and, possibly, pacing, from that electrode to achieve dual chamber sensing/pacing. A fixation mechanism can be attached to the lateral wall of the right atrium to fix the atrial electrode to the lateral wall of the right atrium.

An electrical lead as described herein also would be advantageous for use as an atrial lead in pacemakers and implantable defibrillators. The atrial leads of pacemakers and implantable defibrillators are very susceptible to far-field sensing of non-atrial signals, which often results in errors in rhythm analysis. The bipolar helical electrode described herein can be placed entirely within the myocardium rather than one pole in and one pole out or a bipolar lead partially in, to prevent far-field capture and sensing.

With respect to defibrillation, the bipolar pacing lead described herein can be positioned at the AV septum and used with an ICD lead placed in other cardiac venous structures. The other cardiac venous structures can be specifically selected such that the ICD lead does not cross any cardiac valves. Such structures include, without limitation, the azygos vein, which drains the posterior mediastinium and enters the superior vena cava approximately 3 cm from the junction with the right atrium in its posterior and leftward aspect; an infradiaphragmatic vein that courses over the liver and spleen and under the diaphragm; a leftward hepatic vein, a renal vein and in certain situations in the hemi-azygos vein; and in the more distal (lower) portions of the Inferior Vena Cava (IVC). Placing an ICD lead in any of these venous structures avoids crossing the tricuspid valve and cannulating the coronary sinus. See, for example, Cesario et al. (2004, J. Cardiovasc. Electrophysiol., 15:780-83) and WO 2005/000398 for descriptions of venous ICD lead placement.

The bipolar lead described herein also can be used in the sensing and detection of myocardial infarctions. There has been a renewed interest in developing implantable devices to detect myocardial infarctions in high risk patients. See, for example, U.S. Pat. Nos. 6,468,263; 6,272,379; and 7,107,096. Existing devices, however, all require that the sensing lead be placed at the apex of the right ventricle, which requires that the lead cross the tricuspid valve. Crossing the tricuspid valve has been associated with a number of problems such as regurgitation and/or leaflet damage. The bipolar lead described herein can be implanted without crossing the tricuspid valve and can be used to detect MI.

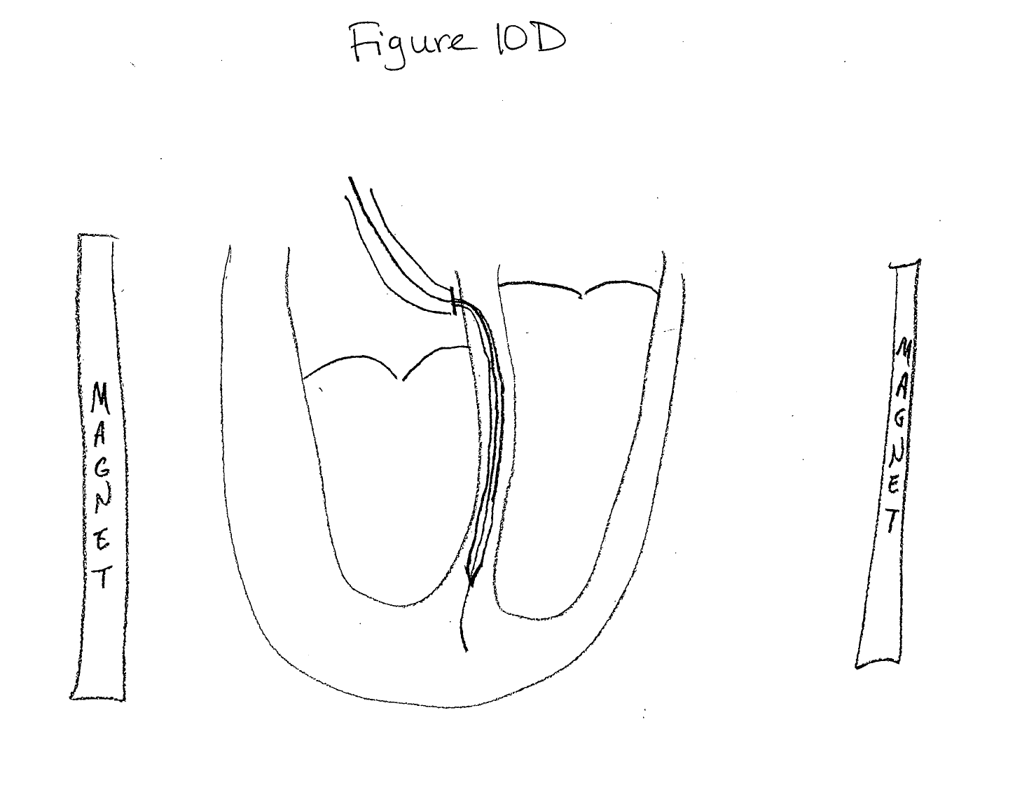

The bipolar leads described herein can be placed at the atrioventricular septum (AVS) as described herein and used to monitor the ST-segment to detect ischemia or myocardial infarction. As indicated herein, positioning the bipolar lead described herein at the AVS does not require crossing the tricuspid valve. Typically, near-field signals accurately detect the timing of local myocardial activation, while far-field signals provide more global ST-segment information. Therefore, choosing a fiducial point for comparison purposes to determine whether ST segments have changed is more difficult with far-field signals and, if signals are not properly aligned, the ST-segments may appear to have shifted, when in fact they have not. To address these issues, near-field electrograms can be used to identify the ST segment, while the ST segment itself can be recorded by far-field electrograms. By taking advantage of two different electrograms (i.e., near-field, which provides reliable timing, and far-field, which provides more ST-segment information globally), unique and reliable observations regarding MI can be made. See, for example, FIG. 9.

With respect to the location of the electrodes, far-field signals can be recorded between a pulse generator shell and a coil in the superior vena cava, an electrode in contact with the myocardium, or an electrode embedded within the myocardium. On the other hand, near-field signals can be recorded between intramyocardial electrograms on the AV septum or elsewhere in the ventricle. In traditional systems (i.e., a non-AV septal system), near-field signals are produced between closely spaced bipoles in the ventricle with an electrode system that is not entirely intramyocardial (i.e., one pole or electrode is in the myocardium and one pole or electrode is outside the myocardium).

ST-segments and morphology differ for different individuals depending upon the exact position of the leads, underlying heart disease, and on body position (laying vs. standing) during the evaluation. In order to compare a current ST-segment with a known normal segment (baseline), a baseline recording of near field and far field segments can be performed and a template generated. Additionally, an accelerometer in the device can determine body position (supine, on side, etc.). Body position can be recorded along with the template, and several “normal” baseline templates created to correspond to various body positions. Following template creation, real-time electrograms can be time-aligned with the template using the near-field signal, and the far field signals compared to determine whether the ST-segments have changed.

Local electrical changes in the ST segment may be meaningful as well as global recordings. Recordings can be obtained from intramyocadial leads alone (near field), between intramyocardial leads, or, in traditional biventricular systems, between the LV to the RV leads (so that these electrodes essentially bracket the left ventricle). Various electrogram vectors can be compared, and the use of multiple vectors can precisely locate the region of ischemia.

The presence of an atrial lead or of electrodes on the AVS lead body permit identification of the timing of atrial activation and, hence, P waves that may occur during the ST segment. This identification can allow those ST segments that occur simultaneously with a P wave to be excluded to avoid erroneously concluding that ST segments had changed when, in fact, the P-wave gave rise to a change. Identifying those P waves that may occur during the ST segment also can allow for subtraction of the P wave to avoid any errors.

In addition to any of the lead placements described herein, intravascular pressure monitoring also can be used to determine whether ST segment changes are associated with hemodynamic sequelae. Lung water can be measured using impedance measurements between an intracardiac electrode in the AVS and a pulse generator. Since thoracic impedance varies with body position, recordings can be correlated with accelerometer recordings of body position as described herein.

The intramyocardial small electrodes described herein, with proper filtering, can function like a monophasic action potential (MAP) catheter to measure action potential duration (e.g., electrical activation on an individual myocyte level, averaged locally; see, for example, U.S. Pat. No. 5,022,396). The action potential duration can be correlated with ST-segment changes and can be useful as a “rate response driver” for an AVS-based CRT pacemaker system. In addition, the action potential (AP) duration can be used for detection of ischemia, changes in the cellular repolarization time, early prediction of malignant ventricular arrhythmia—including those related to anti-arrhythmia medications, inherited conditions, and ischemia. Because an accelerated AP duration is a surrogate for catecholamine levels, the AP duration can be used as an indicator (e.g., a stand-alone indicator) for rate adaptation for pacing.

ST-segments are known to change during pacing, and a paced rhythm is generally considered uninterpretable during ischemia. However, a template can be recorded during intrinsic rhythm and then again during a paced rhythm, and the local (near-field) or regional (near-field) or far field electrogram ST segments can be evaluated to determine if a different response to pacing is observed when the tissue is ischemic versus not ischemic. Thus, the intrinsic and paced electrograms can be compared at baseline (normal) and during periods of questionable ischemia.

This concept of generating a normal template to compare with recordings obtained during ischemic conditions can be performed with standard pacing systems, although the intramyocardial system described herein is likely superior for this purpose. The pacing lead and electrode described herein, by virtue of the intramyocardial bipole and, in addition, specific site pacing at the atrioventricular septum, enhances the ability to perform accurate template matching. With template matching and accurate resting (baseline), local intramyocardial electrogram can be obtained, for example, at implant, and periodically assessed at programmed intervals (e.g., daily, weekly, or monthly), and the sum of a predetermined number of cycles (e.g., about 3 to about 20) can be recorded in device memory. The combination of baseline and intermittent recording of paced and intrinsic electrograms could be utilized for the following clinical scenarios:

a) diagnosis of myocardial infarction. Presently, there is great difficulty with diagnosing acute myocardial infarction or ischemia in patients with pacemakers because of the inherently abnormal depolarization and repolarization pattern for the paced beats. The intramyocardial pacing lead as described herein can be used to compare the intrinsic and paced electrogram at a time at which ischemia and/or infarction is suspected with the template created at baseline and periodically thereafter. This automated comparison can be used to determine the likelihood (e.g., a probability score) that acute ischemia is present. A unique feature of this utility for intramyocardial pacing is the periodic check when pacing is temporarily suspended, which provides information regarding post-pacing (T-wave memory) related changes.

b) diagnosis of proarrhythmia or other malignant arrhythmias. The intramyocardial pacing lead as described herein can be used to monitoring for proarrhythmia or other malignant arrhythmias resulting from nonischemic challenges including membrane active antiarrhythmic agents and dyselectrolytemia.

c) early detection of impending ventricular arrhythmia. Because of the high fidelity electrograms from the intramyocardial bipole and the unique location that allows simultaneous sampling of right and left ventricular tissue, changes in the local electrograms may predict impending arrhythmia (e.g., marked changes in repolarization time) when compared with the automatically generated templates. Significantly differences that are identified may allow intervention prior to the onset of arrhythmia.

AVS pacing is designed to spare the tricuspid valve and to provide resynchronization. At least 50% of patients in CRT device studies have ischemic heart disease. Thus, the ability to screen for recurrent or incipient ischemia adds value to the AVS pacing system by improving its ability to provide diagnostically useful information for patient management.

The advantage of the bipolar lead described herein and methods of using such a lead is that the tricuspid valve does not need to be crossed for implantation, but bipolar sensed electrograms from right ventricular and left ventricular myocardium are obtained. Therefore, the bipolar leads described herein can be combined with pacing and can use electrogram diagnostics (e.g., based on changes in the local electrogram rather than in the surface EKG) to diagnose myocardial ischemia. Having true bipolar sensing from the myocardium itself, which does not occur with traditional leads or by placing leads in the coronary sinus, allows for exact definition of the timing of ST segments.

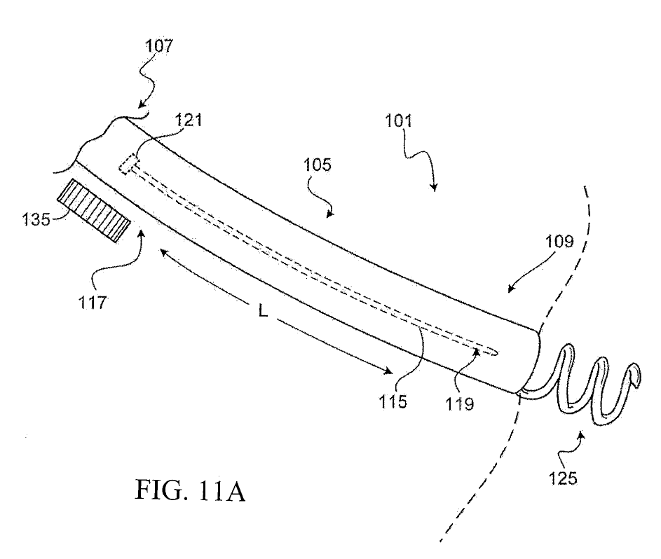

In addition, a bipolar lead as described herein can be introduced into the atrioventricular septum and then, for example, a second electrode (e.g., on a wire) can be tunneled down the interventricular septum to position pacing electrodes and/or ICD coils intramyocardially. FIG. 10 shows a device in which a helical lead first can be introduced at the AV septum, and then a wire containing one or more electrodes, for pacing, sensing or defibrillation, can be tunneled or directed into any number of positions including branching at the base of the heart and extending to either the left or right ventricle (FIG. 10A), exiting into the left ventricle, following along the inner surface of the LV and then anchoring in at the base or wall of the LV (FIG. 10B), or entering from the epicardial surface rather than the endocardial surface (FIG. 10C). In some embodiments, a wire having one or more electrodes attached thereto can be positioned using magnetic guides (e.g., Stereotaxis Magnetic Navigation System, Stereotaxis Corp., St. Louis, Mo.) (FIG. 10D).

With respect to the concept of intramyocardial tunneling as described herein, the bipolar leads described herein are preferred, but traditional leads (e.g., traditional screws or wires) can also be used in such methods. Similar concepts have been described (see, for example, WO 2006/068880, WO 2008/058265 and US 2006/0224224), but all require crossing the TRV, which is completely avoided by the use of the leads and methods described herein.

In addition to the methods already discussed, the leads described herein also can be used for biventricular pacing, which also would not require crossing the tricuspid valve or entering the coronary sinus. For example, to pace the right ventricle, an additional lead can be placed just above the tricuspid valve on the free wall of the tricuspid annulus or anterolaterally. In fact, the right ventricle can be captured along the tricuspid valve annulus (when viewed in the LAO projection, anywhere from approximately the 2 o'clock position up to the 11 o'clock position) without crossing the tricuspid valve. A potential advantage with biventricular stimulation would be that interventricular dyssynchrony (between RV and LV) can be minimized.

### Steerable Sheath or Catheter

A steerable sheath and a steerable catheter are described herein. Uniquely, a steerable sheath such as that described herein allows for active fixation once it is positioned against the target tissue. Fixation of the sheath permits delivery of therapy such as permanent pacing leads to the desired target without risk of tip displacement. The steerable sheath can be “unfixed,” leaving behind a precisely placed permanent lead, or it can be left in place to provide, for example, delivery of additional therapy.

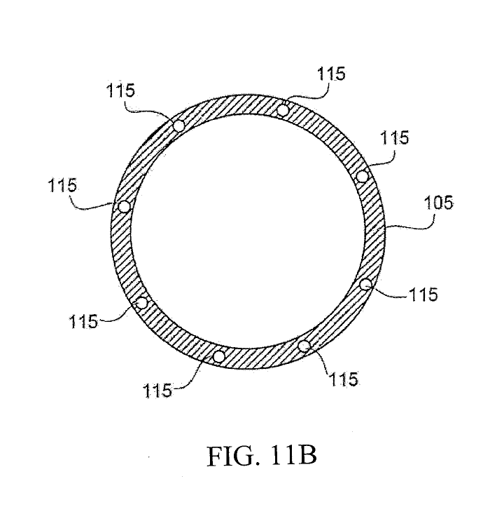

With respect to FIG. 11A, a steerable sheath 101 or a steerable catheter 101 each have a body 105 having a proximal portion 107 and a distal portion 109 along a longitudinal axis L. The sheeth or catheter body 105 is generally tubular and can contain a central lumen (l). The proximal 107 and distal 109 portions of the steerable sheath or catheter 101 can be integrally formed from a biocompatible material having requisite strength and flexibility for introducing and advancing the sheath or catheter into the vasculature of an individual. Appropriate materials are well known in the art and generally include polyamides such as, for example a woven material available from DuPont under the trade name DACRON®.

The steerable sheath or catheter 101 uses longitudinal pins or wires 115 arranged radially around the sheath or catheter to control tip motion (see FIG. 11B). Each longitudinal pin 115 has a proximal end 117 and a distal end 119. The distal ends 119 are attached to the distal portion 109 of the sheath or catheter, and the proximal end 117 of each pin has a pin-control 121. By pushing or pulling on one or more of the pin-controls 121, the distal end of the sheath or catheter 109 can be manipulated for precise navigation by a user. A steerable sheath or catheter 101 is not limited by the number of longitudinal pins 115. The precise number and location of the longitudinal pins 115 will depend on the amount of steerability desired as well as the flexibility of the material used to make the sheath or catheter body 105.

In order to locate the correct anatomical location for placing an electrode, a steerable sheath or steerable catheter 101 can include at least one sensing or imaging component (not shown). The sensing or imaging component can be, without limitation, an ultrasound sensor, a fluoroscopy sensor, a pacing and sensing electrode, and/or a pressure sensor. Using such sensing and/or imaging components, an operator can examiner or determine blood flow and/or supply, an electrogram of the region (e.g., the septum), and/or pressure differences (e.g., in the atrium versus the ventricle). Precise location can also be achieved by extraneous imaging using, for example, fluoroscopy, magnetic guidance, and/or echocardiography. For example, for placement into the atrioventricular septum, a location having a pressure signal indicative of the atrium and an electrical myocardial signal indicative of the right ventricle is identified.

Once the desired location within the heart is located, an anchor screw 125 at the distal portion 109 of the sheath or catheter body 105 can be used to fix the distal portion 109 of the sheath or catheter 101 at the desired location within the heart. Fixation of the distal portion 109 of a sheath via such an anchor screw 125 provides a working channel (or conduit) to the desired location to deliver a pacing lead (e.g., the helical electrode described herein) or other tool (e.g., ablative tools), or to perform tasks such as delivering a therapeutic compound such as drugs or cells or removing a biopsy, while fixation of the distal portion 109 of a catheter via such an anchor screw 125 allows for one or more therapies (e.g., ablative therapy) to be delivered to a precise location without requiring new placement of the catheter each time. In some embodiments, the anchor screw can be an active electrode to permit sensing and/or stimulation (e.g., to determine the responsiveness of the myocardial tissue prior to permanent lead deployment). A steerable sheath having an anchor screw also can be used in procedures such as the Cardiac Contractility Modulation (CCM) in which specific cardiac locations need to be identified. See, for example, Sabbah et al., 2001, Heart Fail. Rev., 6:45-53; and Mohri et al., 2002, Am. J. PhysioL Heart Circ. Physiol., 282:H1642-7.

A steerable sheath or steerable catheter 101 can have one or more control knobs 135 at the proximal portion 107 to screw an anchor screw 125 or a helical electrode 30 in or out of tissue. By turning a control knob 135, for example, clockwise, an operator can screw an anchor screw 125 on a steerable sheath or steerable catheter 101 into tissue at the desired location, or similarly, screw a helical electrode 30 into tissue. Similarly, by turning a control knob 135, for example, counterclockwise, an anchor screw 125 on a steerable sheath or steerable catheter 101 or a helical electrode 30 can be unscrewed from the tissue.

Although the sheath and catheter described herein having an anchor screw at the distal end is described with respect to cardiac applications, it's use is not to be limited in any way. A sheath or a catheter having an anchor screw for fixing its position can be used in, for example, endoscopy in the field of gastrointestinal (GI), urology, or other appropriate fields in medicine or research.

In accordance with the present invention, there may be employed conventional cardiology techniques within the skill of the art. Such techniques are explained fully in the literature.

### EXAMPLES

### Example 1

### Intramyocardial Pacing and Sensing Lead Design

Specially constructed leads with two distal intramyocardial electrodes made of an external helix and a central pin were used for these experiments (FIG. 2). The helix length was 5 mm or 7 mm and constructed of 0.012″ wire. The inner electrode was ⅔^{rd} the length of the outer helix, and constructed of 0.009″ wire. In order to minimize the risk of electrical shorting via mechanical contact between the two electrodes, the inner electrode was partially insulated with 0.001″ polyimide tubing into which 4 (5 mm lead) or 6 (7 mm lead) 0.01″×0.20″ ports were created. The distal electrodes were non-retractable, and actively fixated by rotation of the entire lead body.

### Example 2

### Animal Preparation

Two mongrel dogs weight were placed under general anesthesia and mechanically ventilated. Arterial blood pressure, and surface electrocardiography was continuously recorded. The right internal jugular was cannulated with a 9 Fr sheath. A median sternotomy was performed and the pericardium retracted.

### Example 3

### Experimental Procedure

The aim of the experiment was to: 1) determine whether intramyocardial pacing and sensing is feasible and 2) compare the sensed signals and pacing thresholds of the novel intramyocardial lead with a standard commercially available pacing lead. In the first experiment, epicardial pacing and sensing was undertaken in a carefully controlled manner. Epicardial (rather than endocardial) lead function was initially assessed due to the limited maneuverability of the prototype leads, which, it was anticipated, would limit reliable endocardial positioning. The lead was inserted into the myocardium under direct vision; pacing and sensing was performed at three epicardial locations (right ventricle, lateral left ventricle, and right atrial appendage). The lead was connected to a standard pacing electrophysiology workstation for recording electrograms. Particular attention was paid to defining both atrial and ventricular signals if present and their amplitude. Pacing was performed using a portable pulse generator capable of delivering up to 20 mA current (Medtronic, Inc, Minneapolis, Minn.). Thresholds were assessed at both polarities (cathode central electrode, and cathode helix, for intramyocaridial lead). Evidence of extracardiac stimulation was noted, and during the procedure, any arrythmias or ventricular ectopy were noted. After pacing and sensing experiments were completed, the lead was extracted by employing counterclockwise rotation of the entire lead, and the lead tip and cardiac tissue was examined for damage.

In a second experiment, pacing and sensing function was undertaken in the same manner as before and also compared to that of a standard active fixation pacemaker lead (Flextend Model 4088, Boston Scientific Cardiac Rhythm Management, St. Paul, Minn.). Additionally, the lead was then inserted via the internal jugular vein and advanced into the right ventricular apex under fluoroscopic guidance. The electrode tip was screwed into the endocardium by rotation of the entire lead. After confirming a right ventricular apical location, right ventricular endocardial pacing and sensing was then performed with the intramyocardial lead.

### Example 4

### Results

Ventricular pacing and sensing lead function are summarized in Table 1. For the novel intramyocardial lead, the average R wave at ventricular sites was 7.2 mV, compared to an average R wave of 8.4 mV for the standard active fixation lead placed at identical sites. The average pacing threshold was 0.7 mA at 0.2 msec for the novel lead compared to 1.1 mA for the standard lead. Far field P waves were not recorded on the novel lead; the mean far-field P-wave on the standard lead was 0.7 mV. With the standard lead, phrenic stimulation was seen at threshold (cathode distal) and at 3 mA (cathode proximal electrode). No phrenic stimulation was seen despite outputs up to 20 mA and sites located 3-5 mm from the phrenic nerve.

TABLE 1
Ventricular lead function

| | Atrial | Ventricular | | | |
|---|---|---|---|---|---|
| | electrogram | electrogram | Pacing | | |
| Pacing | amplitude | amplitude | threshold | Phrenic | |
| configuration | (mV) | (mV) | V @ 2 ms | stimulation | Comments |
| Helical RV EPI, | 0 | 8.3 | 0.8 | No | Minimal electrogram |
| tip distal (n = 3) | | | | | decay |
| Helical EPI RV, | 0 | 7.1 | 0.5 | No | |
| tip proximal | | | | | |
| Helixal LV EPI, | 0 | 6.1 | 0.9 | No | Fairly close to phrenic |
| tip distal | | | | | nerve site (3 to 5 mm) |
| Standard LV | 0.5 | 6.7 | 1.1 | Yes (at | Clear but small far field |
| EPI, tip distal | | | | threshold) | atrial electrogram, |
| | | | | | constant phrenic |
| | | | | | stimulation |
| Standard LV | 0.8 | 10 | 1.1 | Yes (at | |
| EPI, tip | | | | 3 mA) | |
| proximal | | | | | |

The results of lead placement at the right atrial appendage base are shown in Table 2. The average atrial electrogram for the intramyocardial lead measured 1.7 mV; far-field R-waves were not present (amplitude 0 mV). Using the standard pacing lead, the atrial electrogram amplitude average measured 1.9 mV; the average far-field R-wave was 1.6 mV. Phrenic stimulation occurred with both polarities using the standard lead, and with neither polarity using the novel intramyocardial lead.

TABLE 2
Atrial lead function

| | Atrial | Ventricular | | | |
|---|---|---|---|---|---|
| | electrogram | electrogram | Pacing | | |
| Pacing | amplitude | amplitude | threshold | Phrenic | |
| configuration | (mV) | (mV) | V @ 2 ms | stimulation | Comments |
| Helical RA EPI | 2.1 | 0 | 0.8 | No | |
| appendage | | | | | |
| base, tip | | | | | |
| distal | | | | | |
| Helical EPI RA | 1.2 | 0 | 0.4 | No | |
| appendage | | | | | |
| base, tip | | | | | |
| proximal | | | | | |
| Standard RA | 1.6 | 2.1 | 1.2 | Yes | Large far field |
| EPI appendage | | | | | ventricular electrogram |
| base, tip distal | | | | | seen |
| Standard RA | 2.1 | 1 | 1 | Yes | |
| EPI appendage | | | | | |
| base, tip | | | | | |
| proximal | | | | | |

There were no complications associated with intramyocardial lead use. Specifically, there was no evidence of electrode short-circuiting, and no evidence of significant myocardial injury (as determined by absence of ectopy, the sensed electrogram, and visual inspection of the myocardium). The screw-in electrodes remained intramyocardial and did not penetrate beyond the epicardium.

These experiments demonstrate that intramyocardial pacing is feasible and results in site-specific pacing and sensing function. The types of leads described herein may eliminate far-field signal oversensing and phrenic stimulation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An electrical lead comprising:
a lead body, wherein said lead body has a proximal end and a distal end, wherein said lead body comprises at least two conductors; and
at least two electrodes, wherein said at least two electrodes are disposed at said distal end of said lead body, wherein said at least two electrodes have a proximal end and a distal end, wherein said proximal ends of said at least two electrodes are electrically connected to said distal ends of said at least two conductors, wherein said at least two electrodes comprise an inner electrode and an outer electrode, wherein said outer electrode is helical shaped and has an inside-the-helix surface and an outside-the-helix surface.

2. The electrical lead of claim 1, wherein said inner electrode is linear shaped.

3. The electrical lead of claim 2, wherein said inner lead is at least partially coated with a non-conductive material.

4. The electrical lead of claim 1, wherein said inner electrode is helical shaped and has an inside-the-helix surface and an outside-the-helix surface.

5. The bipolar pacing lead of claim 1, wherein said inside-the-helix surface of said outer electrode is coated with a non-conductive material.

6. The bipolar pacing lead of claim 1, wherein said outside-the-helix surface of said outer electrode is coated with a non-conductive material.

7. The bipolar pacing lead of claim 4, wherein said inside-the-helix surface of said inner electrode is coated with a non-conductive material.

8. The bipolar pacing lead of claim 4, wherein said outside-the-helix surface of said inner electrode is coated with a non-conductive material.

9. The electrical lead of claim 1, wherein said inner electrode and said outer electrode are co-axial at said proximal ends of said electrodes.

10. The electrical lead of claim 1, wherein the overall length of said electrical lead is about 3 mm to about 7 mm.

11. The electrical lead of claim 1, wherein said conductors are co-axial with one another.

12. The electrical lead of claim 1, wherein said conductors are parallel to one another.

13. The electrical lead of claim 1, wherein said inner electrode and said outer electrode are bipolar.
